(19) United States
(12) Patent Application Publication
(10) Pub. No.: US 2006/0210478 A1
(43) Pub. Date: Sep. 21, 2006

(54) Steady state perfusion methods

(76) Inventor: Robert M. Weisskoff, Lexington, MA (US)

Correspondence Address: FISH & RICHARDSON P.C., PO BOX 1022, MINNEAPOLIS, MN, 55440-1022, US

(21) Appl. No.: 11/346,884

(22) Filed: Feb. 3, 2006

Related U.S. Application Data
(60) Provisional application No. 60/649,713, filed on Feb. 3, 2005.

(51) Int. Cl. A61K 49/00

(52) U.S. Cl. 424/9.36

(57) ABSTRACT

Methods for assessing ischemic coronary artery disease are provided. The methods include administering a contrast agent that binds to a serum protein component to an animal and obtaining an MR image of the animal's myocardium during a period when the animal is experiencing hyperemia.

### CROSS-REFERENCE TO RELATED APPLICATIONS

This application claims priority under 35 U.S.C. § 119(e) to U.S. Provisional Application Ser. No. 60/649,713, filed on Feb. 3, 2005, which is incorporated by reference in its entirety herein.

### TECHNICAL FIELD

This invention relates to MR imaging methods, and more particularly to steady state MR methods for evaluating myocardial perfusion.

### BACKGROUND

About thirteen million Americans suffer from ischemic heart disease (IHD). IHD is often caused by atherosclerosis of the coronary arteries, resulting in restricted blood and oxygen flow to the heart. Common clinical manifestations of IHD include angina, myocardial infarction (heart attack) and cardiac failure.

Diagnosis of IHD ideally would include perfusion and coronary patency information. The most widely used techniques for measuring myocardial perfusion are SPECT (single photon computed tomography) imaging protocols using injectable nuclear agents (e.g., “hot” radiotracers), such as thallium isotope or technetium Sestamibi (MIBI). Frequently the patient is required to undergo a stress test (e.g., a treadmill exercise stress test) to aid in the SPECT evaluation of myocardial perfusion. The cardiac effect of exercise stress can also be simulated pharmacologically by the intravenous administration of a coronary vasodilator. Typically, after injection of the nuclear agent during stress, the myocardium is imaged. A second redistribution rest image is then obtained after an appropriate rest period (approximately 3-4 hours). Alternatively, the patient may be given a second, 2X concentrated dose of the nuclear agent during the rest phase and a second rest image is then acquired. The clinician compares the two image sets to diagnose ischemic areas as “cold” spots on the stress image. SPECT imaging, however, may result in inconclusive perfusion data due to its relatively low sensitivity and specificity.

Recently, magnetic resonance imaging (MRI) techniques have also been proposed to assess myocardial perfusion. In general, MRI is appealing because of its noninvasive character, ability to provide improved spatial resolution, and ability to derive other important measures of cardiac performance, including wall motion and ejection fraction in a single sitting. Current MRI perfusion imaging techniques require rapid imaging of the myocardium during the first pass (after bolus injection) of an extracellular or intravascular MR contrast agent; this technique is referred to as MRFP (magnetic resonance first pass) perfusion imaging. On T1-weighted images, the ischemic zones appear with a delayed and lower signal enhancement (e.g., hypointensity) as compared with normally perfused myocardium. Myocardial signal intensity versus time curves can then be analyzed to extract perfusion parameters. Intensity differences, however, rapidly decrease as the MR contrast agent is diluted in the systemic circulation after the first pass. Furthermore, because of the rapid timing requirement of MRFP perfusion imaging, the patient must undergo pharmacologically-induced stress while positioned inside the MRI apparatus. Rapid imaging may also limit the resolution of the perfusion maps obtained and may result in poor quantification of perfusion.

Because ischemically-injured myocardium contains both reversibly and irreversibly injured regions, accurate characterization of myocardial injury, in particular the differentiation between necrotic (acutely infarcted myocardium), ischemic, and viable myocardial tissue, is an important factor in proper patient management. This characterization can be aided by an analysis of the perfusion and/or reperfusion state of myocardial tissue adjacent to coronary microvessels either before or after an ischemic event (e.g., an acute myocardial infarction).

### SUMMARY

Provided herein are materials and methods for evaluating perfusion, including myocardial perfusion. The methods are performed in the steady-state, thus reducing the technical requirements necessary when imaging is done in the dynamic phase. The use of contrast agents that bind to serum components and exhibit a longer half-life than nonspecific contrast agents allows for both a substantial enhancement in image resolution and a broadened acquisition window.

Accordingly, provided herein is a MR method of assessing the presence or absence of ischemic coronary artery disease that includes:

a) administering intravenously to an animal a MR contrast agent which noncovalently binds to a serum protein component; and

b) obtaining at least one MRI scan of the animal's myocardium during a period when the animal is experiencing a hyperemic response, provided that the at least one hyperemic MRI scan occurs at a time period when the contrast agent is in steady-state equilibrium in the blood of the animal. The at least one hyperemic MRI scan can be obtained at least 3 minutes after intravenous administration of the contrast agent.

In one embodiment, an MR method of assessing the presence or absence of ischemic coronary artery disease includes:

a) administering intravenously to an animal a MR contrast agent which is not covalently bound to a serum protein component; and

b) obtaining at least one MRI scan of said animal's myocardium during a period when said animal is experiencing a hyperemic response, provided that said at least one hyperemic MRI scan occurs at a time period when said contrast agent is in steady-state equilibrium in the blood of said animal. In some cases, the MR contrast agent has a half-life in circulation sufficient to enhance the MR signal of the blood in said animal's myocardium during equilibrium phase of the contrast agent.

Any method described herein can include obtaining at least one MRI scan of an animal's myocardium during a period of rest of the animal, provided that the at least one rest MRI scan occurs at a time period when the contrast agent is in steady-state equilibrium in the blood of the animal.

In certain cases, a serum protein component can be HSA, and a.contrast agent can be MS-325. MS-325 is and does not covalently bind to a serum protein component; MS-325 has a half-life in circulation sufficient to enhance the MR signal of the blood in the myocardium during equilibrium phase. Other examples of such contrast agents are described e.g., in U.S. Pat. No. 6,676,929.

A hyperemic response can be obtained by administering a pharmacologic stress agent to said animal, such as an A_{2A} agonist, or adenosine, dipyridamole, or dobutamine. In other cases, a hyperemic response can be produced by physical stress, e.g., as a result of exercise utilizing a bicycle or a treadmill device.

A method described herein can include comparing the at least one rest MRI scan. to the at least one hyperemic MRI scan and/or can further include obtaining at least one MRI scan of a coronary artery of an animal at any time after step a).

An antidote to a pharmacologic stress agent can be administered to end a hyperemic response, e.g., to allow for the obtaining of a rest MR scan of the myocardium or to end the hyperemia if the procedure is complete. In other cases, the obtaining of rest scans and hyperemic scans (in either order) can be repeated, e.g., by alternating periods of hyperemia with periods of rest (and vice versa). Thus, in certain cases, a method can further include obtaining at least one MR rest scan of an animal's myocardium after administration of an antidote to a pharmacologic stress agent, followed by re-attainment of a hyperemic response, e.g., upon administration of a second dose of a pharmacologic stress agent, followed by the obtaining of least one MRI scan of an animal's myocardium during a second (or subsequent) period of hyperemic response.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the methods, materials, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

Definitions

Commonly used chemical abbreviations that are not explicitly defined in this disclosure may be found in The American Chemical Society Style Guide, Second Edition; American Chemical Society, Washington, DC (1997), “2001 Guidelines for Authors” J. Org. Chem. 66(1), 24A (2001), “A Short Guide to Abbreviations and Their Use in Peptide Science” J. Peptide. Sci. 5, 465-471 (1999).

For the purposes of this application, the term “aliphatic” describes any acyclic or cyclic, saturated or unsaturated, branched or unbranched carbon compound, excluding aromatic compounds.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, the term “alkyl” includes both “unsubstituted alkyls” and “substituted alkyls,” the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes aliphatic groups that may or may not be substituted, as described above for alkyls, containing at least one double bond and at least two carbon atoms. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond and two carbon atoms. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

In general, the term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, such as naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heterocycles,” “heteroaryls,” or “heteroaromatics.” An aryl group may be substituted at one or more ring positions with substituents.

For the purposes of this application, “DTPA” refers to a chemical compound comprising a substructure composed of diethylenetriamine, wherein the two primary amines are each covalently attached to two acetyl groups and the secondary amine has one acetyl group covalently attached according to the following formula:

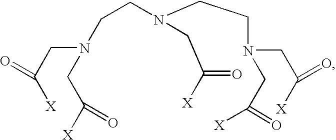

wherein X is a heteroatom electron-donating group capable of coordinating a metal cation, preferably O⁻, OH, NH₂, OPO₃²⁻, or NHR, or OR wherein R is any aliphatic group. When each X group is tert-butoxy (tBu), the structure may be referred to as “DTPE” (“E” for ester).

For the purposes of this application, “DOTA” refers to a chemical compound comprising a substructure composed of 1,4,7,11-tetraazacyclododecane, wherein the amines each have one acetyl group covalently attached according to the following formula:

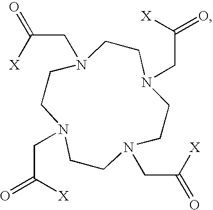

wherein X is defined above.

For the purposes of this application, “NOTA” refers to a chemical compound comprising a substructure composed of 1,4,7-triazacyclononane, wherein the amines each have one acetyl group covalently attached according to the following formula:

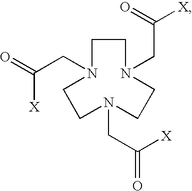

wherein X is defined above.

For the purposes of this application, “DO3A” refers to a chemical compound comprising a substructure composed of 1,4,7,11 -tetraazacyclododecane, wherein three of the four amines each have one acetyl group covalently attached and the dther amine has a substituent having neutral charge according to the following formula:

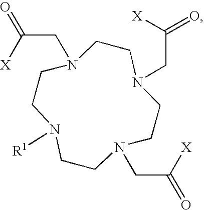

wherein X is defined above and R¹ is an uncharged chemical moiety, preferably hydrogen, any aliphatic, alkyl group, or cycloalkyl group, and uncharged derivatives thereof. The preferred chelate “HP”-DO3A has R¹═—CH₂(CHOH)CH₃.

In each of the four structures above, the carbon atoms of the indicated ethylenes may be referred to as “backbone” carbons. The designation “bbDTPA” may be used to refer to the location of a chemical bond to a DTPA molecule (“bb” for “back bone”). Note that as used herein, bb(CO)DTPA-Gd means a C═O moiety bound to an ethylene backbone carbon atom of DTPA.

The terms “chelating ligand,” “chelating moiety,” and “chelate moiety” may be used to refer to any polydentate ligand which is capable of coordinating a metal ion, including DTPA (and DTPE), DOTA, DO3A, or NOTA molecule, or any other suitable polydentate chelating ligand as is further defined herein, that is either coordinating a metal ion or is capable of doing so, either directly or after removal of protecting groups. The term “chelate” refers to the actual metal-ligand complex, and it is understood that the polydentate ligand will eventually be coordinated to a medically useful metal ion.

The term “specific binding affinity” as used herein, refers to the capacity of a contrast agent or composition (e.g., a small organic molecule) to be taken up by, retained by, or bound to a particular biological component to a greater degree than other components. Contrast agents that have this property are said to be “targeted” to the “target” component. Contrast agents that lack this property are said to be “non-specific” or “non-targeted” agents. The binding affinity of a binding group for a target is expressed in terms of the equilibrium dissociation constant “Kd.”

The term “relaxivity” as used herein, refers to the increase in either of the MRI quantities 1/T1 or 1/T2 per millimolar (mM) concentration of paramagnetic ion or contrast agent, wherein T1is the longitudinal or spin-lattice, relaxation time, and T2 is the transverse or spin-spin relaxation time of water protons or other imaging or spectroscopic nuclei, including protons found in molecules other than water. Relaxivity is expressed in units of mM₋₁s⁻¹.

The terms “target binding” and “binding” for purposes herein refer to non-covalent interactions of a contrast agent with a target. These non-covalent interactions are independent from one another and may be, inter alia, hydrophobic, hydrophilic, dipole-dipole, pi-stacking, hydrogen bonding, electrostatic associations, or Lewis acid-base interactions.

As used herein, all references to “Gd,” “gado,” or “gadolinium” mean the Gd(III) paramagnetic metal ion.

This invention relates to MRI-based methods and contrast agents useful for evaluating myocardial perfusion. Use of the methods and contrast agents can improve the quality of myocardial perfusion maps and provide a more accurate extraction of perfusion parameters. In particular, the invention facilitates the differentiation between necrotic (acutely infarcted myocardium), ischemic, and viable myocardial tissue. In addition, some of the contrast agents of the present invention have an affinity for serum protein components, and can be used to evaluate other physiologic functions or manifestations where such protein components are present in either normal or atypically high concentrations. For example, coronary Magnetic Resonance Angiography (MRA) can be performed with such agents in addition to perfusion imaging.

Contrast Agents

Contrast agents of the invention bind noncovalently to a serum protein component. As a result of such binding, a contrast agent for use in the methods can demonstrate an extended blood half-life as compared to a contrast agent that does not bind to a serum protein component. For example, a contrast agent can bind noncovalently to HSA and demonstrate an extended blood half-life as compared to a nonspecific contrast agent. Methods for determining blood half-life are known to those having ordinary skill in the art; see, e.g., U.S. Pat. No. 6,676,929.

A contrast agent can include one or more physiologically compatible chelating groups (C), a Serum Target Binding Moiety (STBM), and optional linkers (L). The contrast agents target a serum protein component (“the target”) present in the myocardium and bind to it, allowing MR imaging of the target in the myocardium.

A contrast agent may have the following general formula:
[STBM]ₙ−[L]ₘ−[C]ₚ,
where n can range from 1 to 10, m can be 0 to 10, and p can range from 1 to 40.

Certain contrast agents for use in the present methods are described in, e.g., U.S. Pat. No. 6,676,929; U.S. Pat. No. 4,899,755, U.S. Pat. No. 4,880,008, U.S. Publication 20040071705, U.S. Pat. No. 6,803,030, and U.S. Publ. No. 2003/0113265.

For example, the gadolinium chelate of MS-325 as described in U.S. Pat. No. 6,676,929 and having the following structure: 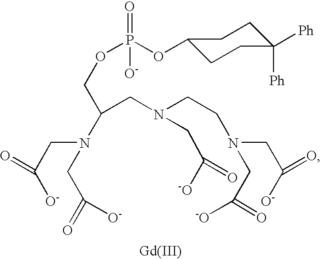
where Ph is phenyl, and pharmaceutically acceptable salts thereof, can be used in the present methods. Other useful contrast agents include gadobenate dimeglumine (known as Multihance), and others as set forth in U.S. Pat. No. 4,916,246 and gadocoletic acid (known as B-22956) and others as described in U.S. Pat. No. 6,803,030. Other contrast agents can be prepared according to the disclosure below.

Serum Target Binding Moiety

Generally, the STBM has an affinity for a serum protein component. For example, the STBM can bind the serum protein component with a dissociation constant of less than 1200 μM (e.g., less than 1000 μM, less than 500 μM, less than 100 μM, or less than 10 μM). In some embodiments, the STBM has a specific binding affinity for a serum protein component relative to a myocardial extracellular matrix component (e.g., a collagen).

Serum protein components include, but are not limited to, serum albumin (e.g., HSA), alpha acid glycoprotein, globulins, fibrinogen, plasminogen, prothrombin, platelets, and lipoproteins. In certain cases, HSA is preferred. A variety of moieties can be used as STBMs. For example, an STBM can be a small organic molecule. A small organic molecule can have a molecular weight of less than about 2000 Daltons, e.g., about 100 to about 750 Daltons. Small organic molecules that include lipophilic and/or amphiphilic organic moieties can be used as STBMs. In certain cases, a “small organic molecule” as used herein can include one to four amino acids, amino acid analogues, nucleosides, and/or nucleotides, or mixtures thereof. Useful STBMs are described in U.S. Pat. No. 6,676,929 (identified as PPBMs therein), U.S. Pat. No. 6,803,030 (identified as bile acids or bile acid residues therein), and U.S. Pat. Publ. 2003/0113265. In other cases, a small organic molecule will include zero amino acids, amino acid analogues, nucleosides, and nucleotides.

In other cases, an STBM can be a peptide or peptidomimetic. A peptide or peptidomimetic can include from about 5 amino acids or amino acid analogues (or combinations thereof) to about 25 amino acids or amino acid analogues (or combinations thereof), and can have a molecular weight from about 600 Daltons to about 3000 Daltons. Certain peptides and peptidomimetics can be from about 10 to about 20 amino acids or amino acid analogues (or combinations thereof).

Peptides, peptidomimetics and small organic molecules can be screened for binding to a serum protein component by methods well known in the art, including equilibrium dialysis, affinity chromatography, and inhibition or displacement of probes bound to the serum protein component.

Metal Chelating Groups

Contrast agents also include a physiologically compatible metal chelating group (C). The C can be any of the many known in the art, and includes, for example, cyclic and acyclic organic chelating agents such as DTPA, DOTA, HP-DO3A, DOTAGA, NOTA, and DTPA-BMA. For MRI, metal chelates such as gadolinium diethylenetriaminepentaacetate (DTPA·Gd), gadolinium tetraamine 1,4,7,10-tetraazacyclododecane-N,N′,N″,N′″-tetraacetate (DOTA·Gd), gadolinium 1,4,7,10-tetraazacyclododecane-1,4,7-triacetate (DO3A·Gd), and bb(CO)DTPA·Gd are particularly useful. In certain embodiments, DOTAGA may be preferred. The structure of DOTAGA, shown complexed with Gd(III), is as follows:

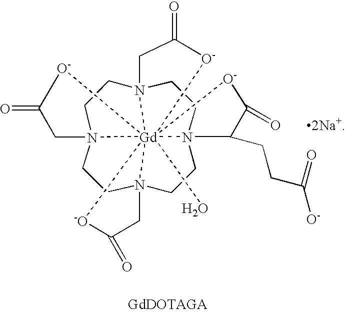

The C can be complexed to a paramagnetic metal ion, including Gd(III), Fe(III), Mn(II), Mn(III), Cr(III), Cu(II), Dy(III), Ho(III), Er(III), Pr(III), Eu(II), Eu(III), Tb(III), Tb(IV), Tm(III), and Yb(III). Additional information regarding C groups and synthetic methodologies for incorporating them into the contrast agents of the present invention can be found in WO 01/09188 and WO 01/08712.

Linkers

In some embodiments, the STBM and the C are covalently bound through a linker (L). The L can include, for example, a linear, branched or cyclic peptide sequence. In one embodiment, a L can include the linear dipeptide sequence G-G (glycine-glycine). In embodiments where the STBM includes a peptide, the L can cap the N-terminus of the MTG peptide, the C-terminus, or both N- and C- termini, as an amide moiety. Other exemplary capping moieties include sulfonamides, ureas, thioureas and carbamates. Ls can also include linear, branched, or cyclic alkanes, alkenes, alkynes, amides, and phosphodiester moieties. The L may be substituted with one or more functional groups, including ketone, ester, amide, ether, carbonate, sulfonamide, or carbamate functionalities. Specific Ls contemplated also include NH—CO—NH—; —CO—(CH₂)ₙ—NH—, where n=1 to 10; dpr; dab; —NH-Ph-; —NH—(CH₂)ₙ—, where n=1 to 10; —CO—NH—; —(CH₂)ₙ—NH—, where n=1 to 10; —CO—(CH₂)ₙ—NH—, where n=l to 10; and —CS—NH—. Additional examples of Ls and synthetic methodologies for incorporating them into contrast agents, particularly contrast agents comprising peptides, are set forth in WO 01/09188 and WO 01/08712.

Properties of Contrast Agents

Contrast agents of the invention can noncovalently bind a serum protein component, such as HSA. For example, at least 10% (e.g., at least 50%, 80%, 90%, 92%, 94%, or 96%) of the contrast agent can be bound to the desired component at physiologically relevant concentrations of contrast agent and target. The extent of binding of a contrast agent to a target can be assessed by a variety of equilibrium binding methods, e.g., ultrafiltration methods; equilibrium dialysis; affinity chromatography; or competitive binding inhibition or displacement of probe compounds.

Contrast agents of the invention can exhibit high relaxivity as a result of target binding (e.g., to HSA), which can lead to better image resolution. The increase in relaxivity upon binding is typically 1.5-fold or more (e.g., at least a 2, 3, 4, 5, 6, 7, 8, 9, or 10 fold increase in relaxivity). Targeted contrast agents having 7-8 fold, 9-10 fold, or even greater than 10 fold increases in relaxivity are particularly useful. Typically, relaxivity is measured using an NMR spectrometer by methods known to those having ordinary skill in the art.

Use of Contrast Agents of the Invention

The methods disclosed herein are useful for monitoring and measuring ischemic coronary artery disease and myocardial perfusion. For example, a method described herein can determine the presence or absence of ischemic coronary artery disease and/or the presence or absence of myocardial infarct. The method can include:

a) administering intravenously to an animal an MR contrast agent which noncovalently binds to a serum protein component, as described previously; and

b) obtaining at least one MRI scan of the animal's myocardium during a period when the animal is experiencing a hyperemic response, provided that the hyperemic MRI scan occurs at a time period when the contrast agent is in steady-state equilibrium in the blood of the animal.

An animal can be any animal, e.g., a human, cat, dog, monkey, cow, horse, sheep, pig, bird, rat, or mouse. Contrast agents for administration can be as described above. In certain cases, MS-325 is administered, as it is known to bind to the serum protein component HSA. As one of skill in the art will recognize, the administered dosage will depend on the contrast agent of interest, the health of the patient, the affinity of the contrast agent for the serum component, the type of MR machine, etc., but typically the dosage will be from about 0.01 to about 0.2 mmol/kg of metal ion (e.g., Gd3+).

As used herein, the term “hyperemia” means the point approaching maximum increased blood supply to an organ or blood vessel for physiologic reasons. A hyperemic response can be exercise-induced or pharmacologically-induced. Exercise-induced peak hyperemia can be achieved through what is commonly known as a “stress test,” (e.g., a treadmill or exercise bike stress test) and has several clinically relevant endpoints, including excessive fatigue, dyspnea, moderate to severe angina, hypotension, diagnostic ST depression, or significant arrhythmia. If exercise is used to induce hyperemia, the animal can, in certain cases, exercise for at least one additional minute after hyperemia is obtained before the obtaining of the hyperemic MR scan.

The cardiac effect of exercise-induced peak hyperemia can also be simulated pharmacologically. For example, in certain cases the hyperemic response is obtained by administering a pharmacologic stress agent to the animal, such as an A_{2A} agonist. In other cases, a pharmacologic stress agent is selected from adenosine, dipyridamole, and dobutamine.

During the period of hyperemia, one or more MR scans of the animal's myocardial tissue can be obtained, provided that the administered contrast agent has reached steady-state equilibrium. As used herein, “steady-state equilibrium” means that a contrast agent has achieved equilibrium in the blood of an animal (e.g., a human), meaning that it has been thoroughly mixed with the blood of the patient. It should be noted that the term “steady-state equilibrium” is not meant to imply that the concentration of the contrast agent remains constant after administration, as one of skill in the art will recognize that the contrast agent will be removed from circulation and excreted over time. Instead, the term steady-state equilibrium is meant to reflect that the contrast agent has been well-mixed in the blood of the animal and that the concentration is homogeneous in the blood in the imaging volume, and thus that a concentration gradient of the agent is not generally present in the blood. Thus, while first-pass imaging relies on a concentration gradient in the blood to track, e.g., a bolus of contrast agent in the blood, the present methods take place after such a bolus has been dispersed throughout the blood of the patient.

Generally, the acquisition of the MR image begins in a time frame at least 4-5 times greater than that required for a first pass distribution of the contrast agent. In humans, with a bolus venous injection of a contrast agent, the bolus typically passes through the right heart after approximately 12 sec., and through the left heart after about another 12 sec. Thus, from time of injection to the first pass of the agent through the entire heart, approximately 24-30 seconds have passed. The second pass of the contrast agent usually is seen approximately 45 sec. later.

Steady-state equilibrium, therefore, is typically reached after about 120 seconds. Accordingly, the MR scan can be performed after about 180 seconds (3 minutes), or about 210 seconds, or about 240 seconds (4 minutes), or about 270 seconds, or about 300 seconds (5 minutes). In certain cases, because the contrast agents for use in the methods described herein bind noncovalently to a serum protein component, they exhibit extended blood half-lives. As such, an MRI scan done can be performed after about 5 to about 10 mins. after administration, e.g., after about 10, 15, 20, 25, 30, 45, 60 minutes, about 1.5 hours, or even about 2 hours after administration of the contrast agent. Thus, for example, MS-325 can be administered and imaging can be performed at a time period of about 5 minutes to 2 hours, or more preferably about 10 minutes to about 1 hour, after administration.

An MR image of the myocardial tissue of the animal in the hyperemic state can be compared with an MR image of the myocardial tissue taken when the animal is at rest. A rest MR image can be acquired either before the induction of hyperemia or after the hyperemia has abated. For example, an antidote to a pharmacologic stress agent can be administered to end a hyperemic response, the animal can cease exercising for an appropriate period of time, or adenosine administration is stopped, and a rest MR image can be obtained. In other cases, a rest MR image can be obtained before the induction of hyperemia. In certain cases in using pharmacologic stress agents, periods of hyperemia and rest can be repeated using a pharmacologic stress agent antidote to obtain multiple MR images and/or scans of the myocardium during rest and hyperemia.

The rest MR scan can be performed at a time period when the contrast agent is also in steady-state equilibrium in the blood. For example, an animal can be administered a contrast agent and a rest scan can be obtained once the contrast agent has reached steady-state equilibrium in the blood, e.g., at a time period as outlined previously. Subsequently, hyperemia can be induced, and a hyperemic scan obtained (e.g., while the contrast agent remains in steady-state equilibrium). Zones of abnormal, or low, perfusion will be hypointense (less intense) compared to normal myocardium in the hyperemia image. An assessment of the degree or severity of ischemic coronary artery disease can be made based on the extent (e.g. size) and relative hypointensity of the ischemic zones. In addition, methods disclosed herein can determine the location and severity of coronary artery disease, ischemic heart disease, and the presence or absence of myocardial infarct.

Because certain of the contrast agents for use in the methods exhibit extended blood half-lives, MRA methods (e.g., to assess coronary artery stenosis and patency) can be performed either before or after the described perfusion methods. MRA methods using, e.g., MS-325, are known in the art; see, e.g., Radiology (December 2003) 229(3):811-20 (Epub 2003 Oct. 30). MRA methods using Multihance are also known; see, e.g., Eur. Radiology (Nov. 2003) Vol. 13 Suppl 3: N19-27; J. Magn. Res. Imaging (March 2004) 19(3):261-73.

Certain MR techniques and pulse sequences may be preferred in the methods of the invention. Examples of desirable pulse sequences include cardiac gated 2d spin echo (TE/TR=15/1RR) sequences, T₁ weighted spoiled echo gradient sequences (cardiac gated, flip/TE/TR=30°/2/8), IR-prepped gradient echo sequences, and navigated IR-prepped sequences. Other T₁ weighted sequences may also be used that are well known to those skilled in the art, e.g., sequences to image normally perfused myocardium. Similarly, those of skill in the art will recognize other suitable MR-based methods for detecting infarct, e.g., T2 weighted imaging, delayed ECS imaging, and myocardial imaging.

Methods may be used that involve the acquisition and/or comparison of contrast-enhanced and non-contrast images and/or the use of one or more additional contrast agents. For example, methods as set forth in U.S. Pat. No. 6,549,798 and U.S. Publication US-2003-0028101-A maybe used.

Pharmaceutical Compositions

Contrast agents and compositions of the invention can be formulated as a pharmaceutical composition in accordance with routine procedures. As used herein, the contrast agents or compositions of the invention can include pharmaceutically acceptable derivatives thereof. “Pharmaceutically acceptable” means that the agent can be administered to an animal without unacceptable adverse effects. A “pharmaceutically acceptable derivative” means any pharmaceutically acceptable salt, ester, salt of an ester, or other derivative of a contrast agent or compositions of this invention that, upon administration to a recipient, is capable of providing (directly or indirectly) a contrast agent or composition of this invention or an active metabolite or residue thereof. Other derivatives are those that increase the bioavailability when administered to a mammal (e.g., by allowing an orally administered compound to be more readily absorbed into the blood) or which enhance delivery of the parent compound to a biological compartment (e.g., the brain or lymphatic system) thereby increasing the exposure relative to the parent species. Pharmaceutically acceptable salts of the contrast agents or compositions of this invention include counter ions derived from pharmaceutically acceptable inorganic and organic acids and bases known in the art.

Pharmaceutical compositions of the invention can be administered by any route, including both oral and parenteral administration. Parenteral administration includes, but is not limited to, subcutaneous, intravenous, intraarterial, interstitial, intrathecal, and intracavity administration. When administration is intravenous, pharmaceutical compositions may be given as a bolus, as two or more doses separated in time, or as a constant or non-linear flow infusion. Thus, compositions of the invention can be formulated for any route of administration.

Typically, compositions for intravenous administration are solutions in sterile isotonic aqueous buffer. Where necessary, the composition may also include a solubilizing agent, a stabilizing agent, and a local anesthetic such as lidocaine to ease pain at the site of the injection. Generally, the ingredients will be supplied either separately, e.g. in a kit, or mixed together in a unit dosage form, for example, as a dry lyophilized powder or water free concentrate. The composition may be stored in a hermetically sealed container such as an ampule or sachette indicating the quantity of active agent in activity units. Where the composition is administered by infusion, it can be dispensed with an infusion bottle containing sterile pharmaceutical grade “water for injection,” saline, or other suitable intravenous fluids. Where the composition is to be administered by injection, an ampule of sterile water for injection or saline may be provided so that the ingredients may be mixed prior to administration. Pharmaceutical compositions of this invention comprise the contrast agents of the present invention and pharmaceutically acceptable salts thereof, with any pharmaceutically acceptable ingredient, excipient, carrier, adjuvant or vehicle.

A contrast agent is preferably administered to the patient in the form of an injectable composition. The method of administering a contrast agent is preferably parenterally, meaning intravenously, intra-arterially, intrathecally, interstitially or intracavitarilly. Pharmaceutical compositions of this invention can be administered to mammals including humans in a manner similar to other diagnostic or therapeutic agents.

### EXAMPLES

### Example 1

### Pie Study of Perfusion Using MS-325 at Steady State

A domestic pig (approx 50 kg B.W.) is anesthetized and intubated. The animal undergoes surgical intervention to partially occlude the distal portion of the left circumflex coronary artery (LCX). A calibrated angioplasty balloon is delivered by catheter, guided by X-ray fluoroscopy, from the femoral artery to the heart. It is advanced into the left circumflex coronary artery and inflated to create the equivalent of an 80-90% stenosis. The balloon catheter will remain inflated and at a constant inflation pressure for the duration of the imaging procedure, to simulate a static lesion and stenosis in the coronary artery.

The pig is then transported to the MRI suite and remains under general anesthesia and intubated for the duration of the imaging examination. Sufficient MRI scout scans to plan the myocardial imaging are acquired. Then 0.05 mmol/kg of MS-325 is administered as a single intraveneous injection. Enough time (ca. 10 minutes) for the agent to achieve equilibrium in the blood elapses before imaging commences.

Perfusion imaging is performed using a saturation-recovery gradient echo methods in order to sensitize the MRI to the lowered T1 of the imaging agent. Three short-axis slices (7.5 mm, with 7.5 mm slice separations) are acquired, so that 16 of the 17 AHA/ACC myocardial segments can be visualized. In this implementation, cardiac-triggering is employed to control heart motion, and breathing is suspended to eliminate diaphragmatic motion. Image data is acquired during mid-diastole of each heartbeat, and imaging lasts approximately 45 seconds.

Analysis of MR images demonstrated a suspicious hypo-intense region in the anterior wall of the left ventrical. Vasodilatory stress is then induced with a constant infusion of 0.25 mg/kg/min adenosine. After 5 minutes of adenosine application, imaging is repeated while the adenosine application persists. The corresponding image during stress shows a greater degree of negative contrast with the remaining myocardial wall, confirming a perfusion deficit consistent with obstruction of the left circumflex coronary artery.

### Example 2

A domestic pig (approx 60 kg B.W.) is anesthetized and intubated. The animal undergoes surgical intervention to partially occlude the distal portion of the left circumflex coronary artery (LCX). A calibrated angioplasty balloon is delivered by catheter, guided by X-ray fluoroscopy, from the femoral artery to the heart. It is advanced into the Left Circumflex coronary artery and inflated to create the equivalent of an 80-90% stenosis. The balloon catheter will remain inflated and at a constant inflation pressure for the duration of the imaging procedure, to simulate a static lesion and stenosis in the coronary artery.

The pig is then transported to the MRI suite and remains under general anesthesia and intubated for the duration of the imaging examination. Sufficient MRI scout scans to plan the myocardial imaging are aquired. 0.05 mmol/kg of MS-325 is administered as a single intraveneous injection. Enough time (ca. 10 minutes) for the agent to achieve equilibrium in the blood elapses before perfusion imaging commences.

Perfusion imaging is performed using a gradient echo method in order to sensitize the MRI to the lowered T1 of the imaging agent (TR=3.2 ms, FA=12°). Three short-axis 10 mm slices are acquired, so that 16 of the 17 AHA/ACC myocardial segments can be visualized. In this implementation, cardiac-triggering is employed to control heart motion and breathing is suspended to eliminate diaphragmatic motion. Image data (106 phase-step resolution) is acquired once during mid-diastole of each heartbeat. A time-series of 360 images are acquired over approximately 5 minutes.

Vasodilatory stress is induced with a constant infusion of 0.25 mg/kg/min adenosine. Analysis of MR images demonstrates a hypo-intense region in the wall of the left ventricle, indicating a perfusion deficit, which is confirmed by measurements of fluorescent microspheres injected during imaging and analyzed post-mortem.

### Example 3

A domestic pig (approx 60 kg B.W.) is anesthetized and intubated. The animal undergoes surgical intervention to partially occlude the distal portion of the left circumflex coronary artery (LCX). A calibrated angioplasty balloon is delivered by catheter, guided by X-ray fluoroscopy, from the femoral artery to the heart. It is advanced into the Left Circumflex coronary artery and inflated to create the equivalent of an 80-90% stenosis. The balloon catheter will remain inflated and at a constant inflation pressure for the duration of the imaging procedure, to simulate a static lesion and stenosis in the coronary artery.

The pig is then transported to the MRI suite and remains under general anesthesia and intubated for the duration of the imaging examination. Sufficient MRI scout scans to plan the myocardial imaging are aquired. 0.05 mmol/kg of MS-325 is administered as a single intraveneous injection. Enough time (ca. 10 minutes) for the agent to achieve equilibrium in the blood elapses before perfusion imaging.

Perfusion imaging is performed using a gradient echo method in order to sensitize the MRI to the lowered T1 of the imaging agent (TR=5.0 ms, FA=12°). Three 10 mm short-axis slices are acquired, so that 16 of the 17 AHA/ACC myocardial segments can be visualized. In this implementation, cardiac-triggering is employed to control heart motion and breathing is suspended to eliminate diaphragmatic motion. Data is acquired over multiple heartbeats, so that 4 sets of image data with 189 phase-step resolution is acquired for each of the three slices over approximately 2 minutes, and averaged to create 3 low noise/high resolution images.

Vasodilatory stress is then induced with a constant infusion of 0.25 mg/kg/min adenosine. Imaging is repeated during the adenosine stress. Analysis of the MR images demonstrates a hypo-intense region in the myocardial wall, indicating a perfusion deficit that is confirmed by measurements of fluorescent microspheres injected during imaging and analyzed post-mortem.

A number of embodiments of the invention have been described. Nevertheless, it will be understood that various modifications may be made without departing from the spirit and scope of the invention.

What is claimed is:

1. An MR method of assessing the presence or absence of ischemic coronary artery disease comprising:
a) administering intravenously to an animal a MR contrast agent which noncovalently binds to a serum protein component; and
b) obtaining at least one MRI scan of said animal's myocardium during a period when said animal is experiencing a hyperemic response, provided that said at least one hyperemic MRI scan occurs at a time period when said contrast agent is in steady-state equilibrium in the blood of said animal.

2. The method of claim 1, wherein said at least one hyperemic MRI scan is obtained at least 3 minutes after said intravenous administration of said contrast agent.

3. The method of claim 1, further comprising obtaining at least one MRI scan of said animal's myocardium during a period of rest of said animal, provided that said at least one rest MRI scan occurs at a time period when said contrast agent is in steady-state equilibrium in the blood of said animal.

4. The method of claim 1, wherein said serum protein component is HSA.

5. The method of claim 1, wherein said contrast agent is MS-325.

6. The method of claim 1, wherein about 0.01 to about 0.2 mmol/kg of said contrast agent is injected.

7. The method of claim 1, wherein said hyperemic response is obtained by administering a pharmacologic stress agent to said animal.

8. The method of claim 7, wherein said pharmacologic stress agent is an A_{2A} agonist.

9. The method of claim 7, wherein said pharmacologic stress agent is selected from adenosine, dipyridamole, and dobutamine.

10. The method of claim 1 wherein the hyperemic response is produced by physical stress.

11. The method of claim 10, wherein said physical stress is the result of exercise utilizing a bicycle or a treadmill device.

12. The method of claim 3, further comprising comparing the at least one rest MRI scan to the at least one hyperemic MRI scan.

13. The method of claim 1, further comprising obtaining at least one MRI scan of a coronary artery of said animal at any time after step a).

14. The method of claim 1, further comprising determining the degree or severity of ischemic coronary artery disease.

15. The method of claim 7, wherein an antidote to the pharmacologic stress agent is administered to end the hyperemic response.

16. The method of claim 15, wherein at least one MRI rest scan of said animal's myocardium is obtained after said administration of said antidote, wherein a hyperemic response in said animal is re-attained upon administration of a second dose of a pharmacologic stress agent, and wherein at least one MRI scan of said animal's myocardium is obtained during said second period of hyperemic response.

17. An MR method of assessing the presence or absence of ischemic coronary artery disease comprising:
a) administering intravenously to an animal a MR contrast agent which is not covalently bound to a serum protein component; and
b) obtaining at least one MRI scan of said animal's myocardium during a period when said animal is experiencing a hyperemic response, provided that said at least one hyperemic MRI scan occurs at a time period when said contrast agent is in steady-state equilibrium in the blood of said animal.
